(19) United States
(12) Patent Application Publication
(10) Pub. No.: US 2026/0216891 A1
(43) Pub. Date: Jul. 30, 2026

(54) ROBOT SYSTEM AND ROBOT

(71) Applicant: KAWASAKI JUKOGYO KABUSHIKI KAISHA, Kobe-shi, Hyogo (JP)

(72) Inventors: Hiroki KOKUSHI, Kobe-shi (JP); Mitsunobu OKA, Kobe-shi (JP)

(73) Assignee: KAWASAKI JUKOGYO KABUSHIKI KAISHA, Kobe-shi, Hyogo (JP)

(21) Appl. No.: 18/878,930

(22) PCT Filed: Jun. 14, 2023
(86) PCT No.: PCT/JP2023/022095

§ 371 Date: Dec. 26, 2024

(30) Foreign Application Priority Data
Jun. 30, 2022 (JP) 2022-105455

(51) Int. Cl. B25J 13/08; A61B 6/04; A61B 50/13; B25J 9/00; B25J 9/16

(52) U.S. Cl. CPC B25J 13/085; A61B 6/0407; A61B 50/13; B25J 9/0009; B25J 9/1664

(57) ABSTRACT

A robot system (100) includes a controller (40) configured or programmed to perform at least one of a control to attach a robot main body (20) to a tabletop (10) based on a position of the tabletop (10) detected by a position detector (30) or a control to detach the tabletop (10) from the robot main body (20) based on a position of a mobile cart (300) detected by the position detector (30).

### TECHNICAL FIELD

The present disclosure relates to a robot system and a robot.

### BACKGROUND ART

Conventionally, a robot system including a tabletop on which a patient is placed is disclosed. For example, Japanese Patent Laid-Open No. 2012-005557 discloses a medical robot system including a surgical robot, a movable bed, and an imaging diagnostic device. In Japanese Patent Laid-Open No. 2012-005557, the movable bed is non-removably fixed to a station that supports the movable bed, and the position, orientation, and posture of the movable bed can be changed on the station. The imaging diagnostic device is spaced apart from the surgical robot. When a patient is treated using the medical robot system, the patient is first placed on the movable bed. The movable bed then moves into an imaging area of the imaging diagnostic device. The imaging diagnostic device then images the patient. The movable bed then moves close to the surgical robot. The surgical robot then performs treatment on the patient.

### PRIOR ART

### Patent Document

Patent Document 1: Japanese Patent Laid-Open No. 2012-005557

### SUMMARY OF THE INVENTION

In the medical robot system described in Japanese Patent Laid-Open No. 2012-005557, when the patient is imaged by the imaging diagnostic device, it is necessary to position the patient on a top plate of the movable bed in advance. In Japanese Patent Laid-Open No. 2012-005557, the movable bed is non-removably fixed to the station, and thus the next patient to be treated cannot be positioned on the top plate until the treatment of the patient currently positioned on the top plate is completed and the patient is removed from the top plate. It takes a relatively long time to position the patient on the top plate. This reduces the throughput of the medical robot system. Therefore, it is desired to improve the throughput of the medical robot system. The throughput refers to the amount of processing per unit time.

The present disclosure is intended to solve the above problem. The present disclosure aims to provide a robot system and a robot each capable of improving throughput.

A robot system according to a first aspect of the present disclosure includes a tabletop on a mobile cart to allow a patient to be placed thereon, a robot main body including a robot arm to be detachably attached to the tabletop, a position detector on the robot main body to detect at least one of a position of the tabletop on the mobile cart or a position of the mobile cart without the tabletop placed thereon, and a controller. The controller is configured or programmed to perform at least one of a control to move the robot main body based on the position of the tabletop on the mobile cart, which is detected by the position detector, to attach the robot main body to the tabletop or a control to move the robot main body based on the position of the mobile cart without the tabletop placed thereon, which is detected by the position detector, to place the tabletop attached to the robot main body on the mobile cart to detach the tabletop from the robot main body.

As described above, the robot system according to the first aspect of the present disclosure includes the controller configured or programmed to perform at least one of a control to move the robot main body based on the position of the tabletop on the mobile cart, which is detected by the position detector, to attach the robot main body to the tabletop or a control to move the robot main body based on the position of the mobile cart without the tabletop placed thereon, which is detected by the position detector, to place the tabletop attached to the robot main body on the mobile cart to detach the tabletop from the robot main body. Accordingly, the tabletop can be attached and detached to and from the robot main body, and thus while treatment is performed on the patient placed on the tabletop attached to the robot main body, the next patient to be treated can be positioned on the tabletop on another mobile cart. Consequently, the throughput of the robot system can be improved. Furthermore, the controller can correct the amount of movement of the robot main body even when the position of the tabletop on the mobile cart deviates from a position taught to the robot main body in advance by performing a control to move the robot main body based on the position of the tabletop on the mobile cart, detected by the position detector. Consequently, the robot main body can be appropriately attached to the tabletop. Furthermore, the controller can correct the amount of movement of the robot main body even when the position of the mobile cart deviates from a position taught to the robot main body in advance by performing a control to move the robot main body based on the position of the mobile cart detected by the position detector. Consequently, the tabletop attached to the robot main body can be appropriately on the mobile cart. Furthermore, the position detector is on the robot main body, and thus the position detector moves together with the robot main body. Therefore, unlike a case in which the position detector is fixed at a position spaced apart from the robot main body, it is possible to reduce or prevent the possibility that the position detector cannot detect an object to be detected, such as the tabletop or the mobile cart, due to the object to be detected being hidden behind the robot main body. Consequently, at least one of a control to attach the robot main body to the tabletop or a control to detach the tabletop from the robot main body can be accurately performed.

A robot according to a second aspect of the present disclosure includes a robot main body including a robot arm to be detachably attached to a tabletop on a mobile cart to allow a patient to be placed thereon, a position detector on the robot main body to detect a position of the tabletop on the mobile cart or a position of the mobile cart without the tabletop placed thereon, and a controller. The controller is configured or programmed to perform at least one of a control to move the robot main body based on the position of the tabletop on the mobile cart, which is detected by the position detector to attach the robot main body to the tabletop on the mobile cart, or a control to move the robot main body based on the position of the mobile cart without the tabletop placed thereon, which is detected by the position detector, to place the tabletop attached to the robot main body on the mobile cart to detach the tabletop from the robot main body.

As described above, the robot according to the second aspect of the present disclosure includes the controller configured or programmed to perform at least one of a control to move the robot main body based on the position of the tabletop on the mobile cart, which is detected by the position detector, to attach the robot main body to the tabletop or a control to move the robot main body based on the position of the mobile cart without the tabletop placed thereon, which is detected by the position detector, to place the tabletop attached to the robot main body on the mobile cart to detach the tabletop from the robot main body. Accordingly, the tabletop can be attached and detached to and from the robot main body, and thus while treatment is performed on the patient placed on the tabletop attached to the robot main body, the next patient to be treated can be positioned on the tabletop on another mobile cart. Consequently, the throughput of the robot system can be improved. Furthermore, the controller can correct the amount of movement of the robot main body even when the position of the tabletop on the mobile cart deviates from a position taught to the robot main body in advance by performing a control to move the robot main body based on the position of the tabletop on the mobile cart, detected by the position detector. Consequently, the robot main body can be appropriately attached to the tabletop. Furthermore, the controller can correct the amount of movement of the robot main body even when the position of the mobile cart deviates from a position taught to the robot main body in advance by performing a control to move the robot main body based on the position of the mobile cart detected by the position detector. Consequently, the tabletop attached to the robot main body can be appropriately on the mobile cart. Furthermore, the position detector is on the robot main body, and thus the position detector moves together with the robot main body. Therefore, unlike a case in which the position detector is fixed at a position spaced apart from the robot main body, it is possible to reduce or prevent the possibility that the position detector cannot detect an object to be detected, such as the tabletop or the mobile cart, due to the object to be detected being hidden behind the robot main body. Consequently, it is possible to provide the robot capable of accurately performing at least one of a control to attach the robot main body to the tabletop or a control to detach the tabletop from the robot main body.

According to the present disclosure, the throughput of the robot system or the robot can be improved.

### BRIEF DESCRIPTION OF THE DRAWINGS

FIG. 1 is a block diagram showing the configuration of a robot system according to an embodiment.

FIG. 2 is a perspective view showing the configuration of the robot system according to the embodiment.

FIG. 3 is a diagram showing the lower surface of a tabletop according to the embodiment.

FIG. 4 is a side view of a mobile cart as viewed from the Y2 side.

FIG. 5 is a top view of the mobile cart.

FIG. 6 is a side view of the mobile cart as viewed from the Y1 side.

FIG. 7 is a perspective view showing the configuration of a robot main body according to the embodiment.

FIG. 8 is a side view showing a base member, a position detector, the tabletop, and automatic tool changers.

FIG. 9 is a top view showing the base member, the position detector, and the automatic tool changers.

FIG. 10 is a flowchart of the operation of the robot system according to the embodiment performed when the tabletop is attached.

FIG. 11 is a diagram for illustrating the operation of a horizontal sensor to detect first detection target members.

FIG. 12 is a diagram for illustrating the pitch angle of the tabletop.

FIG. 13 is a top view of the mobile cart appropriately positioned.

FIG. 14 is a diagram for illustrating the yaw angle of the mobile cart.

FIG. 15 is a diagram showing the robot main body moved below the tabletop.

FIG. 16 is a diagram showing the tabletop attached to the robot main body.

FIG. 17 is a diagram showing the tabletop lifted by the robot main body.

FIG. 18 is a flowchart of the operation of the robot system according to the embodiment performed when the tabletop is detached.

FIG. 19 is a diagram showing the robot main body moved above the tabletop.

FIG. 20 is a diagram showing the tabletop placed on the mobile cart.

FIG. 21 is a diagram showing the tabletop detached from the robot main body.

FIG. 22 is a block diagram showing the configuration of a robot according to a modified example.

### MODES FOR CARRYING OUT THE INVENTION

An embodiment embodying the present disclosure is hereinafter described on the basis of the drawings.

The configuration of a robot system 100 according to this embodiment is now described. In this specification, an upward-downward direction is defined as a Z direction. The upper side is defined as a Z1 direction, and the lower side is defined as a Z2 direction. A direction perpendicular to the Z direction is defined as an X direction. In the X direction, one side is defined as an X1 side, and the other side is defined as an X2 side. A direction perpendicular to the Z direction and the X direction is defined as a Y direction. In the Y direction, one side is defined as a Y1 side, and the other side is defined as a Y2 side.

### Robot System

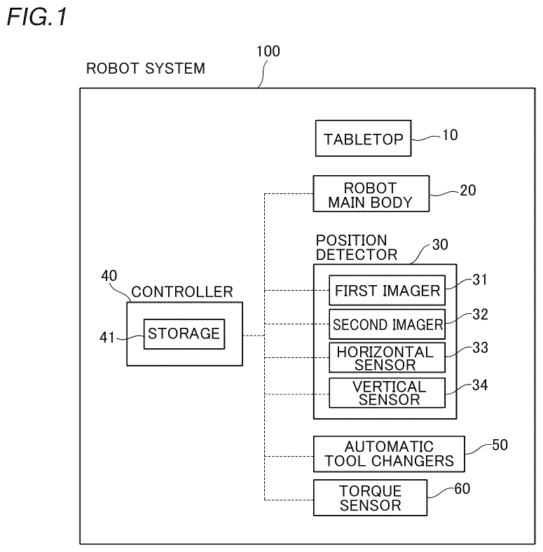

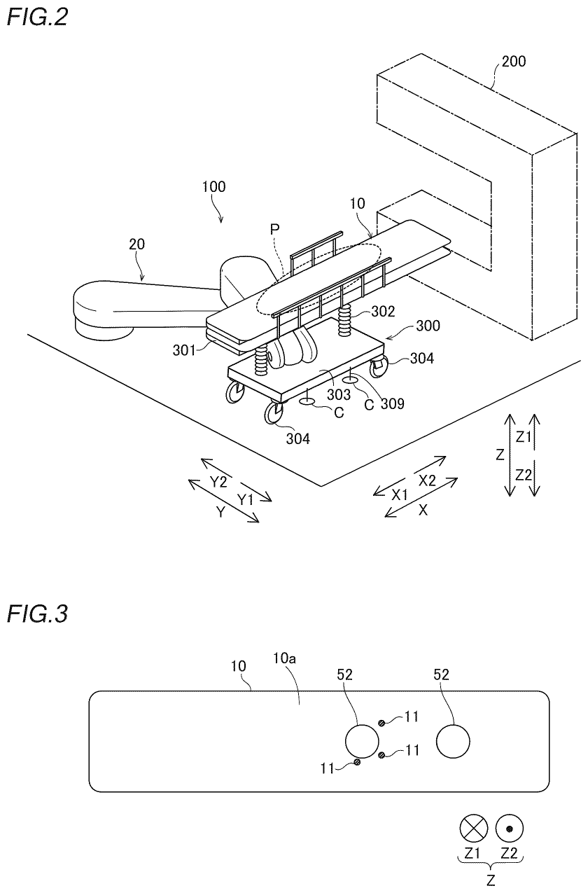

As shown in FIG. 1, the robot system 100 includes a tabletop 10, a robot main body 20, a position detector 30, a controller 40, automatic tool changers 50, and a torque sensor 60. As shown in FIG. 2, the robot system 100 is arranged in the vicinity of an inspection device 200. The robot system 100 moves the tabletop 10, on which a patient P is placed and which is placed on a mobile cart 300, to the inspection device 200. After the examination is completed, the robot system 100 moves the tabletop 10, on which the patient P is placed, from the inspection device 200 to the mobile cart 300.

As shown in FIG. 2, the patient P is positioned on the tabletop 10 placed on a mounting table 301 of the mobile cart 300. With the patient P positioned in this state, an operator moves the mobile cart 300 to the vicinity of the robot system 100. A plurality of circles C are drawn on a floor surface on which the robot system 100 is placed. A plurality of chains 309 hang down from the mobile cart 300 to the floor surface. The operator moves the mobile cart 300 such that the chains 309 of the mobile cart 300 are positioned inside the circles C.

### Tabletop

The patient P is placed on the tabletop 10. The tabletop 10 is placed on the mobile cart 300. Specifically, the tabletop 10 has a rectangular shape. In FIG. 2, the longitudinal direction of the tabletop 10 is defined as the X direction, and the short-side direction of the tabletop 10 is defined as the Y direction. The tabletop 10 is placed on the mobile cart 300.

In this embodiment, as shown in FIG. 3, the tabletop 10 includes first markers 11. The first markers 11 are arranged on the lower surface 10a of the tabletop 10. Specifically, a plurality of first markers 11 are arranged.

### Mobile Cart

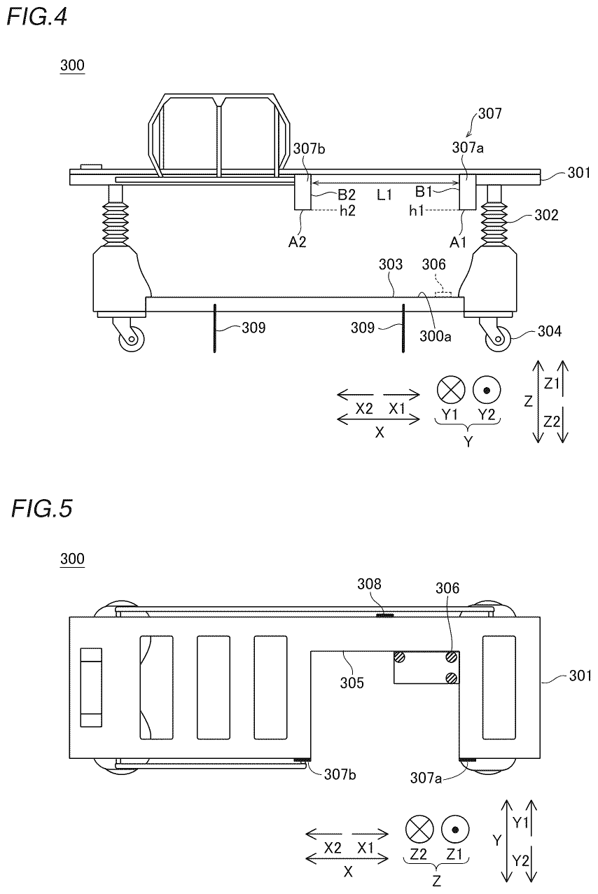

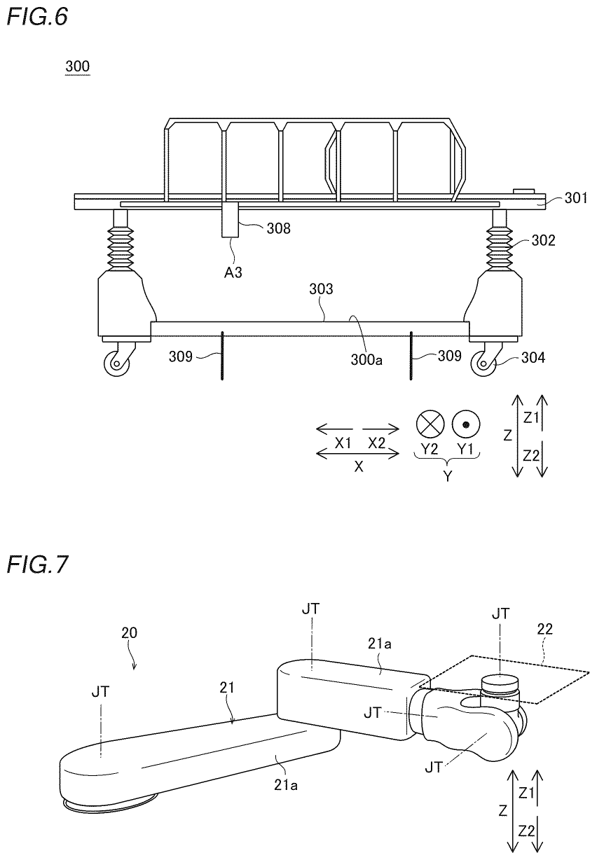

As shown in FIG. 4, the mobile cart 300 includes the mounting table 301, lifters 302, a base 303, and wheels 304. As shown in FIG. 5, the mobile cart 300 includes a notch 305, a second marker 306, and first detection target members 307. As shown in FIG. 6, the mobile cart 300 includes a second detection target member 308. The tabletop 10 is placed on the mounting table 301. A pair of lifters 302 are arranged. The lifters 302 support both ends of the mounting table 301 in the X direction. The lifters 302 raise and lower the mounting table 301. For example, the lifters 302 include gas cylinders, and the mounting table 301 is raised and lowered by the gas cylinders. The base 303 supports the lifters 302. A plurality of wheels 304 are arranged. For example, four wheels 304 are arranged. The wheels 304 are attached to the base 303.

As shown in FIG. 5, the notch 305 is formed in the mounting table 301. As viewed from the Z1 side, the notch 305 has a rectangular shape. A robot arm 21 passes through the notch 305 when moving from the Z2 side to the Z1 side of the mounting table 301.

In this embodiment, the second marker 306 is arranged on the upper surface 300a of the mobile cart 300. The second marker 306 is arranged at a position visible from the Z1 side of the mobile cart 300. For example, the second marker 306 is arranged on the upper surface 300a of the base 303. The second marker 306 is exposed from the notch 305 as viewed from the Z1 side.

As shown in FIG. 4, a pair of first detection target members 307 are arranged. The first detection target members 307 are arranged at both ends of the notch 305. The pair of first detection target members 307 are referred to as a first detection target member 307a and a first detection target member 307b. The first detection target member 307a is arranged at an end of the notch 305 on the X1 side, and the first detection target member 307b is arranged at an end of the notch 305 on the X2 side. As shown in FIG. 5, the first detection target member 307a and the first detection target member 307b are arranged at an end of the mounting table 301 on the Y2 side. The shapes of the first detection target member 307a and the first detection target member 307b are rectangular and flat, for example. As shown in FIG. 4, when the mounting table 301 is arranged along a horizontal plane, the height h1 of a lower end A1 of the first detection target member 307a is the same as the height h2 of a lower end A2 of the first detection target member 307b.

As shown in FIG. 6, for example, one second detection target member 308 is arranged. The second detection target member 308 is arranged at the end of the mounting table 301 on the Y2 side. As shown in FIG. 5, the second detection target member 308 is arranged between the pair of first detection target members 307, as viewed from the Y1 side.

### Robot Main Body

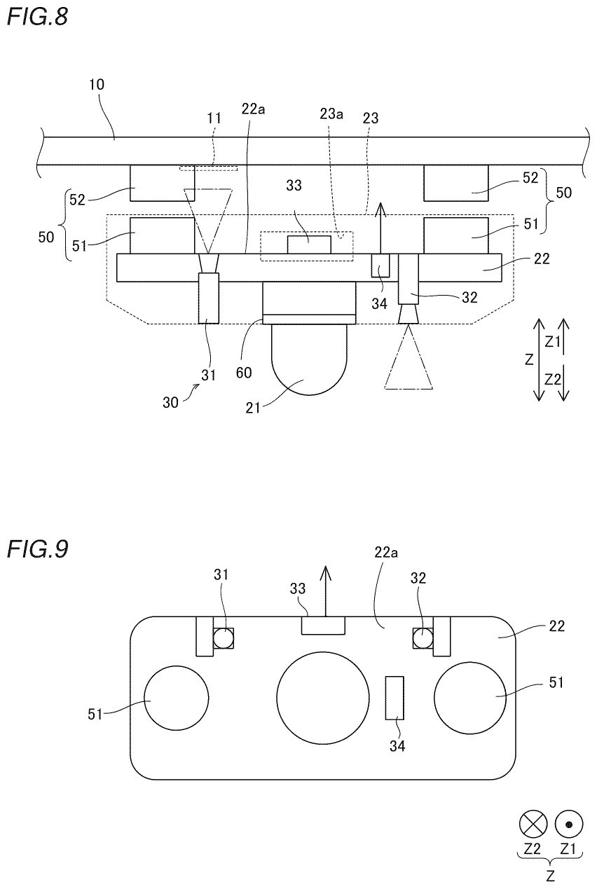

As shown in FIG. 7, the robot main body 20 includes the robot arm 21 and a base member 22. As shown in FIG. 8, the robot main body 20 includes a housing 23. The robot arm 21 is detachably attached to the tabletop 10. The robot arm 21 is an articulated robot arm. The robot arm 21 includes a plurality of arms 21a. The robot arm 21 includes a plurality of rotation axes JT.

As shown in FIG. 8, the base member 22 is attached to a distal end of the robot arm 21. The base member 22 is fixed to the robot arm 21. The base member 22 rotates around a rotation axis JT at the most distal end of the robot arm 21. The base member 22 is arranged in the housing 23. The housing 23 includes a hole 23a to allow detection light emitted from a horizontal sensor 33 described below to pass therethrough.

### Position Detector

In this embodiment, the position detector 30 detects at least one of the position of the tabletop 10 placed on the mobile cart 300 or the position of the mobile cart 300 without the tabletop 10 placed thereon. The position detector 30 is arranged on the robot main body 20. The position detector 30 includes a first imager 31, a second imager 32, the horizontal sensor 33, and a vertical sensor 34.

The first imager 31 images the first markers 11 on the tabletop 10 placed on the mobile cart 300. The first imager 31 is a two-dimensional camera, for example. The first imager 31 is placed on the base member 22. The first imager 31 is placed on the base member 22 so as to face the Z1 side. The first imager 31 images the first markers 11 arranged on the lower surface 10a of the tabletop 10 from the Z2 side of the tabletop 10.

The second imager 32 images the second marker 306 on the mobile cart 300 without the tabletop 10 placed thereon. The second imager 32 is a two-dimensional camera, for example. The second imager 32 is placed on the base member 22. The second imager 32 is placed on the base member 22 so as to face the Z2 side. The second imager 32 images the second marker 306 arranged on the upper surface 300a of the mobile cart 300 from the Z1 side of the mobile cart 300.

As shown in FIG. 8, the horizontal sensor 33 detects the position of an object to be measured in a horizontal direction. For example, the horizontal sensor 33 detects a distance along the horizontal direction to the object to be measured. The horizontal sensor 33 detects the positions of the first detection target members 307 in the horizontal direction. The horizontal sensor 33 detects the position of the second detection target member 308 in the horizontal direction. The horizontal sensor 33 is a displacement sensor, for example. The horizontal sensor 33 emits detection light along the horizontal direction and detects reflected light reflected from the object to be measured. The horizontal sensor 33 is placed on the base member 22. The horizontal sensor 33 is placed on the base member 22 so as to face the horizontal direction. As shown in FIG. 9, the horizontal sensor 33 is placed on the upper surface 22a of the base member 22.

As shown in FIG. 8, the vertical sensor 34 detects a distance along a vertical direction to the object to be measured. The vertical sensor 34 detects a distance along the vertical direction to the tabletop 10. The vertical sensor 34 is a reflective distance sensor, for example. The vertical sensor 34 is placed on the base member 22. The horizontal sensor 33 is placed on the base member 22 so as to face the Z1 side. When the vertical sensor 34 is a reflective distance sensor, detection light is emitted from the vertical sensor 34 toward the Z1 side. As shown in FIG. 9, the vertical sensor 34 is placed on the upper surface 22a of the base member 22.

### Automatic Tool Changers

As shown in FIG. 8, the automatic tool changers 50 attach and detach the tabletop 10 to and from the robot main body 20. The automatic tool changers 50 include master-side devices 51 and slave-side devices 52. The master-side devices 51 are placed on the base member 22. Specifically, the master-side devices 51 are placed on the upper surface 22a of the base member 22. The slave-side devices 52 are placed on the tabletop 10. Specifically, the slave-side devices 52 are placed on the lower surface 10a of the tabletop 10. A plurality of automatic tool changers 50 are arranged. For example, two automatic tool changers 50 are arranged.

### Torque Sensor

The torque sensor 60 is placed on the robot main body 20. The torque sensor 60 detects a force applied to the robot main body 20 due to a load when the robot main body 20 is attached to the tabletop 10. Specifically, the torque sensor 60 is arranged between the robot arm 21 and the base member 22.

The controller 40 controls the entire robot system 100. In this embodiment, the controller 40 performs a control to move the robot main body 20 based on the position of the tabletop 10 placed on the mobile cart 300, which is detected by the position detector 30, to attach the robot main body 20 to the tabletop 10. The controller 40 performs a control to move the robot main body 20 based on the position of the mobile cart 300 without the tabletop 10 placed thereon, which is detected by the position detector 30, to place the tabletop 10 attached to the robot main body 20 on the mobile cart 300 to detach the tabletop 10 from the robot main body 20. In other words, the controller 40 performs both a control to attach the robot main body 20 to the tabletop 10 and a control to detach the tabletop 10 from the robot main body 20, based on the detection result of the position detector 30. The controller 40 includes a central processing unit (CPU), a read-only memory (ROM), a random access memory (RAM), etc., for example.

### Operation of Robot System Performed When Tabletop is Attached

The operation of the robot system 100 performed when the tabletop is attached is now described. The mobile cart 300 with the patient P positioned thereon has been moved in advance to the vicinity of the robot system 100 by the operator.

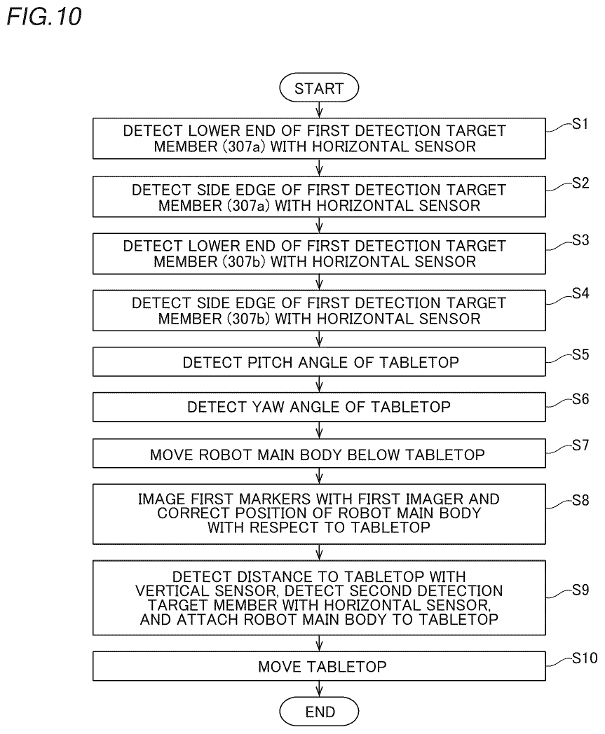

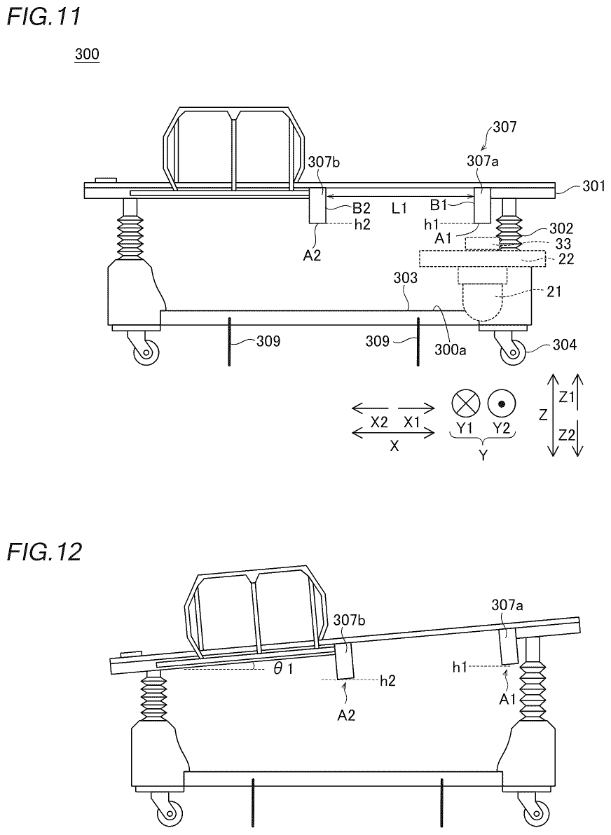

In this embodiment, as shown in FIG. 10, in step S1, the controller 40 controls the horizontal sensor 33 to detect the position of the first detection target member 307a in the horizontal direction. The controller 40 detects the height h1 of the lower end A1 of the first detection target member 307a based on the detection result of the horizontal sensor 33. As shown in FIG. 11, the controller 40 moves the robot arm 21 from the Z2 side to the Z1 side. Detection light is emitted from the horizontal sensor 33 along a Y1 direction. When the height of the horizontal sensor 33 coincides with the height h1 of the lower end A1 of the first detection target member 307, the detection light emitted from the horizontal sensor 33 is reflected by the first detection target member 307a. The horizontal sensor 33 detects the reflected light from the first detection target member 307a. The controller 40 detects the height h1 of the lower end A1 of the first detection target member 307a based on the horizontal sensor 33 detecting the reflected light. The controller 40 detects a distance to the first detection target member 307a along the Y direction based on the horizontal sensor 33 detecting the reflected light.

In step S2, the controller 40 moves the robot arm 21 horizontally toward the X2 side. The controller 40 detects the position of an end B1 of the first detection target member 307a on the X2 side based on the detection light emitted from the horizontal sensor 33 being no longer reflected by the first detection target member 307a such that the reflected light from the first detection target member 307a is no longer detected. The position of the end B1 of the first detection target member 307a on the X2 side coincides with the position of the end of the notch 305 on the X1 side.

In step S3, similarly to step S1, the controller 40 controls the horizontal sensor 33 to detect the position of the first detection target member 307b in the horizontal direction. The controller 40 detects the height h2 of the lower end A2 of the first detection target member 307b based on the detection result of the horizontal sensor 33. Specifically, the controller 40 moves the robot arm 21 from the Z2 side to the Z1 side. The controller 40 detects the height h2 of the lower end A2 of the first detection target member 307b based on the horizontal sensor 33 detecting the reflected light from the lower end A2 of the first detection target member 307. The controller 40 detects a distance to the first detection target member 307b along the Y direction based on the horizontal sensor 33 detecting the reflected light.

In step S4, similarly to step S2, the controller 40 moves the robot arm 21 horizontally toward the X1 side. The controller 40 detects the position of an end B2 of the first detection target member 307b on the X1 side based on the reflected light from the first detection target member 307b being no longer detected. The position of the end B2 of the first detection target member 307b on the X1 side coincides with the position of the end of the notch 305 on the X2 side.

In this embodiment, in step S5, the controller 40 detects the tilt angle θ1 of the tabletop 10 placed on the mobile cart 300 with respect to the horizontal plane based on the detected height h1 of the lower end A1 of the first detection target member 307a and the detected height h2 of the lower end A2 of the first detection target member 307b. The tilt angle θ1 with respect to the horizontal plane is called a pitch angle. Specifically, the controller 40 detects the tilt angle θ1 of the mounting table 301 of the mobile cart 300 based on a difference between the detected height h1 of the lower end A1 of the first detection target member 307a and the detected height h2 of the lower end A2 of the first detection target member 307b. When the mounting table 301 is aligned with the horizontal plane, the difference between the height h1 and the height h2 is zero. As shown in FIG. 12, when the mounting table 301 is inclined with respect to the horizontal plane, the difference between the height h1 and the height h2 is not zero. The tilt angle θ1 of the mounting table 301 changes depending on the magnitude of the difference between the height h1 and the height h2. The controller 40 detects the tilt angle θ1 of the mounting table 301 based on the difference between the height h1 and the height h2. The tilt angle θ1 of the mounting table 301 is the same as the tilt angle θ1 of the tabletop 10.

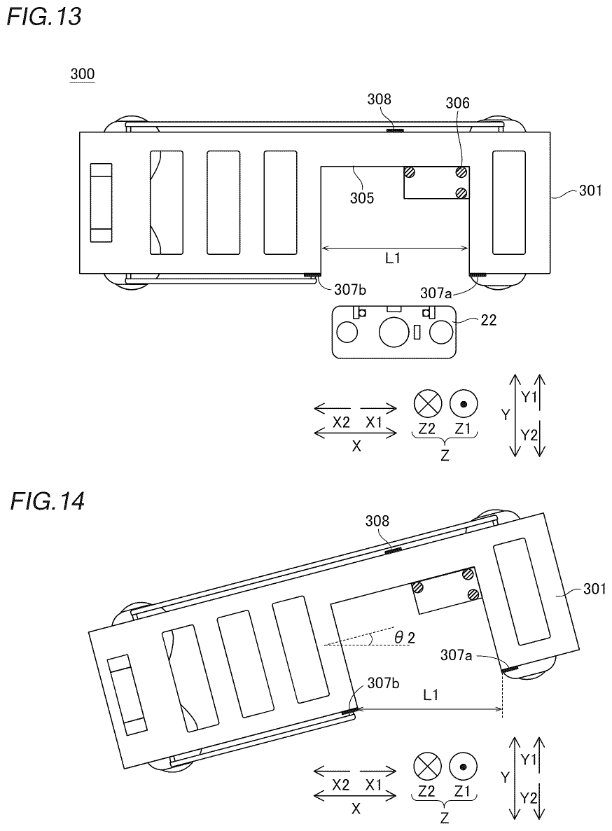

In this embodiment, in step S6, the controller 40 detects an interval L1 between the pair of first detection target members 307a and 307b based on the detection result of the horizontal sensor 33. Then, the controller 40 detects the rotation angle θ2 of the tabletop 10 in the horizontal plane based on the detected interval L1. As shown in FIG. 13, when the mobile cart 300 is arranged along the X direction, the interval L1 detected by the controller 40 is increased. On the other hand, as shown in FIG. 14, when the mobile cart 300 rotates around an axis along the Z direction, the interval L1 detected by the controller 40 is decreased. The rotation angle θ2 of the mobile cart 300 around the axis along the Z direction is called a yaw angle. The interval L1 changes depending on the magnitude of the yaw angle. The controller 40 detects the rotation angle θ2 of the mounting table 301 of the mobile cart 300 around the axis along the Z direction based on the interval L1. The rotation angle θ2 of the mounting table 301 around the axis along the Z direction is the same as the rotation angle θ2 of the tabletop 10 about the axis along the Z direction.

The controller 40 may detect the rotation angle θ2 of the tabletop 10 placed on the mobile cart 300 around the axis along the Z direction based on a difference between the distance to the first detection target member 307a along the Y direction detected in step S1 and the distance to the first detection target member 307b along the Y direction detected in step S3. Specifically, as shown in FIG. 13, when the mobile cart 300 is arranged along the X direction, the difference between the distance to the first detection target member 307a and the distance to the first detection target member 307b is zero. On the other hand, as shown in FIG. 14, when the mobile cart 300 is rotating around the axis along the Z direction, the difference between the distance to the first detection target member 307a and the distance to the first detection target member 307b is not zero. The difference between the distance to the first detection target member 307a and the distance to the first detection target member 307b changes depending on the magnitude of the yaw angle.

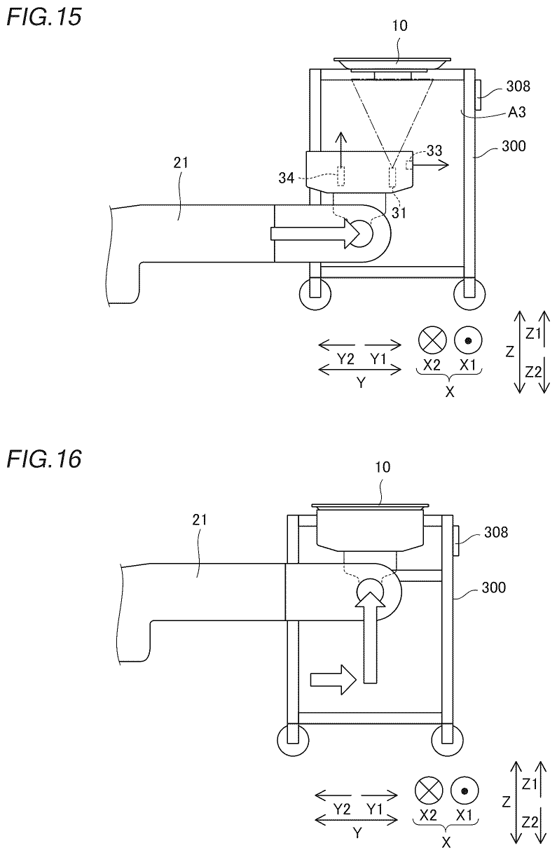

In step S7, as shown in FIG. 13, the controller 40 performs a control to move the robot main body 20 to the Z2 side of the notch 305 of the mobile cart 300 on which the tabletop 10 is placed. Specifically, as shown in FIG. 15, the controller 40 performs a control to move the robot main body 20 to the Z2 side of the notch 305 of the mounting table 301 based on the tilt angle θ1 of the mounting table 301 and the rotation angle θ2 of the mounting table 301 around the axis along the Z direction. The movement path of the robot main body 20 to the Z2 side of the notch 305 is stored in advance in a storage 41 of the controller 40. The controller 40 corrects the movement path of the robot main body 20 based on the tilt angle θ1 of the mounting table 301 and the rotation angle θ2 of the mounting table 301 around the axis along the Z direction such that the robot main body 20 does not contact the mobile cart 300.

In step S8, the controller 40 controls the first imager 31 to image the first markers 11 on the tabletop 10 placed on the mobile cart 300. The controller 40 then corrects the position of the robot main body 20 with respect to the tabletop 10 based on information captured by the first imager 31. Specifically, the position of the robot main body 20 with respect to the tabletop 10 is stored in advance in the storage 41 of the controller 40. The controller 40 corrects a position to which the robot main body 20 actually moves based on the image of the first markers 11 captured by the first imager 31. The controller 40 corrects the positions of the robot main body 20 in the X direction and the Y direction based on the image of the first markers 11. The controller 40 detects the first markers 11 from the image captured by the first imager 31 by image processing.

The controller 40 also performs a control to correct the position of the robot main body 20 with respect to the tabletop 10 based on the image of the first markers 11 captured by the first imager 31, and at least one of the detected tilt angle θ1 of the tabletop 10 or the rotation angle θ2 of the tabletop 10 around the axis along the Z direction. In this embodiment, the position of the robot main body 20 is corrected based on all of the image of the first markers 11, the tilt angle θ1 of the tabletop 10, and the rotation angle θ2 of the tabletop 10 around the axis along the Z direction. Specifically, the position of the robot main body 20 is corrected such that the tabletop 10 and the base member 22 are parallel to each other. The position of the robot main body 20 is corrected such that the rotation angle θ2 of the tabletop 10 around the axis along the Z direction is equal to the rotation angle θ2 of the base member 22 around the axis along the Z direction.

In step S9, the controller 40 performs a control to attach the robot main body 20 to the tabletop 10. Specifically, the controller 40 performs a control to attach the robot main body 20 to the tabletop 10 placed on the mobile cart 300 while detecting a distance to the tabletop 10 along the vertical direction with the vertical sensor 34. For example, the controller 40 moves the robot main body 20 to the Z1 side while controlling the moving speed of the robot main body 20 according to the distance to the tabletop 10 along the vertical direction detected with the vertical sensor 34.

The controller 40 also performs a control to attach the robot main body 20 to the tabletop 10 placed on the mobile cart 300 while determining whether or not the relative positions of the robot main body 20 and the mobile cart 300 in the horizontal plane are normal based on the result of detection of the second detection target member 308 by the horizontal sensor 33 and while detecting the distance to the tabletop 10 along the vertical direction with the vertical sensor 34. Specifically, the controller 40 determines that the tilt angle θ1 of the tabletop 10 and the rotation angle θ2 of the tabletop 10 around the axis along the Z direction are within the allowable ranges based on reflected light from the second detection target member 308 being detected by the horizontal sensor 33. When the tilt angle θ1 of the tabletop 10 and the rotation angle θ2 of the tabletop 10 are within the allowable ranges, the controller 40 performs a control to attach the robot main body 20 to the tabletop 10. Furthermore, while the robot main body 20 is moved to the Z1 side, the controller 40 determines that a height at which the robot main body 20 is attached to the tabletop 10 is normal based on the lower end A3 of the second detection target member 308 being detected by the horizontal sensor 33. When the height at which the robot main body 20 is attached to the tabletop 10 is not normal, the controller 40 stops a control to attach the robot main body 20 to the tabletop 10.

As shown in FIG. 16, the controller 40 performs a control to attach the robot main body 20 to the tabletop 10 by controlling the automatic tool changers 50. The controller 40 performs a control to connect the master-side devices 51 placed on the base member 22 to the slave-side devices 52 placed on the tabletop 10.

The controller 40 performs a control to adjust the position of the robot main body 20 based on the force applied to the robot main body 20 detected by the torque sensor 60 while the robot main body 20 and the tabletop 10 contact each other, and attach the robot main body 20 to the tabletop 10 placed on the mobile cart 300. In other words, the controller 40 performs a control to finely adjust the position of the robot main body 20 based on the force applied to the robot main body 20 detected by the torque sensor 60.

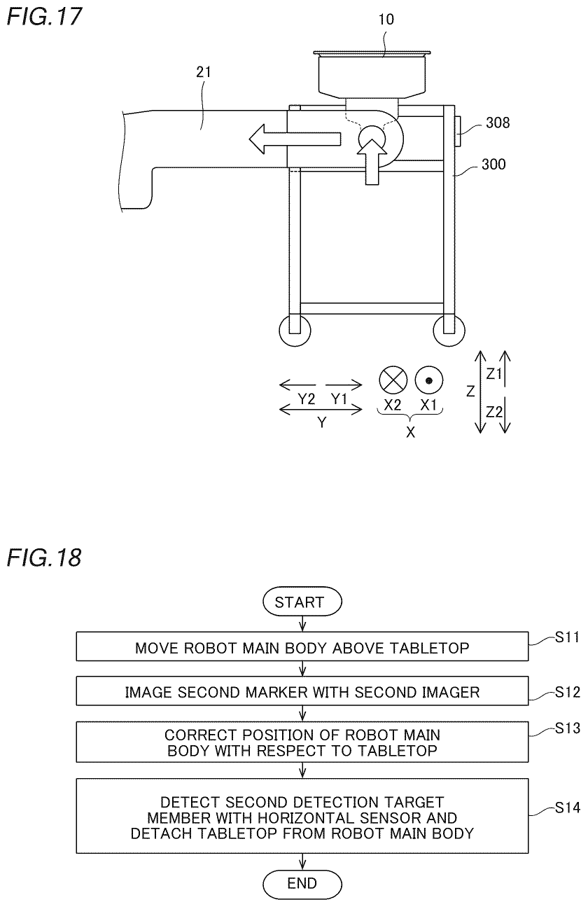

In step S10, as shown in FIG. 17, the controller 40 moves the tabletop 10 attached to the robot main body 20 to the Z1 side and also moves it to the inspection device 200.

### Operation of Robot System Performed When Tabletop is Detached

The operation of the robot system 100 performed when the tabletop is detached is now described. The tabletop 10 is attached to the robot main body 20. The mobile cart 300 without the tabletop 10 placed thereon is placed in advance in the vicinity of the robot system 100.

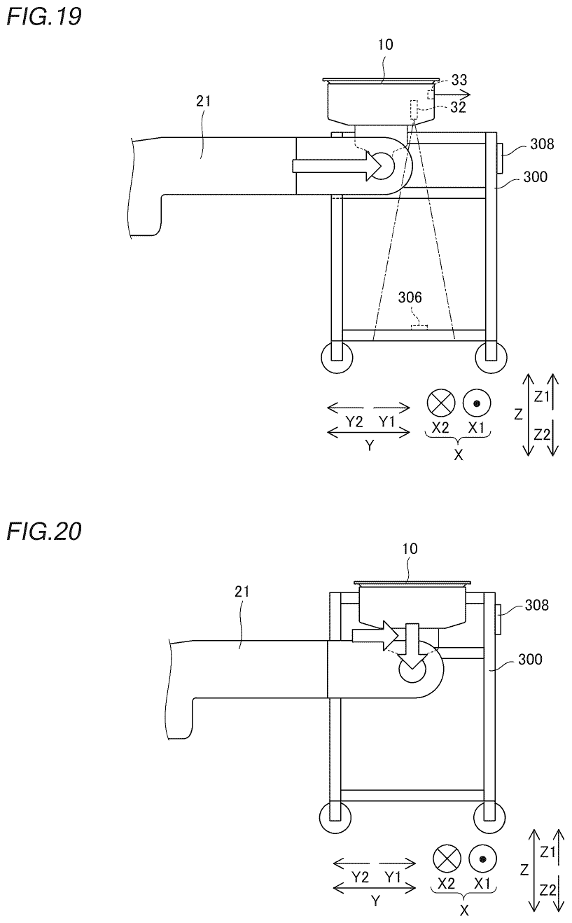

In step S11 shown in FIG. 18, the controller 40 performs a control to move the robot main body 20 to which the tabletop 10 has been attached to the Z1 side of the notch 305 of the mobile cart 300, as shown in FIG. 19.

In step S12, the controller 40 controls the second imager 32 to image the second marker 306 on the mobile cart 300.

In step S13, the controller 40 performs a control to correct the position of the robot main body 20 with respect to the mobile cart 300 based on the image of the second marker 306 captured by the second imager 32. Specifically, the controller 40 corrects the positions of the robot main body 20 in the X direction and the Y direction with respect to the mobile cart 300 based on the image of the second marker 306 captured by the second imager 32. The controller 40 detects the second marker 306 from the image captured by the second imager 32 by image processing.

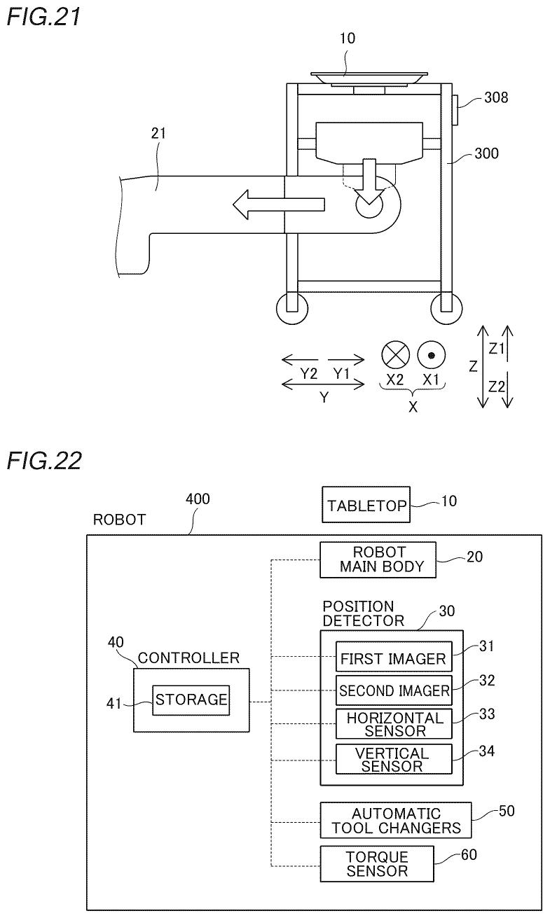

In step S14, as shown in FIG. 20, the controller 40 performs a control to place the tabletop 10 attached to the robot main body 20 on the mobile cart 300, and detach the tabletop 10 from the robot main body 20, as shown in FIG. 21. Specifically, the controller 40 performs a control to lower the robot main body 20 from the Z1 side toward the Z2 side of the notch 305 of the mobile cart 300. The controller 40 also performs a control to lower the robot main body 20 while determining whether or not the relative positions of the robot main body 20 and the mobile cart 300 in the horizontal plane are normal based on the result of detection of the second detection target member 308 by the horizontal sensor 33. With the tabletop 10 placed on the mounting table 301 of the mobile cart 300, the controller 40 performs a control to detach the master-side devices 51 placed on the base member 22 from the slave-side devices 52 placed on the tabletop 10 by controlling the automatic tool changers 50. The controller 40 moves the robot main body 20 to a position spaced apart from the mobile cart 300. After that, the operator moves the mobile cart 300 to a predetermined location. Furthermore, the operator moves another mobile cart 300, on which the next patient P to be examined is placed, to the vicinity of the robot system 100. Then, the controller 40 performs a control to attach the tabletop 10 to the robot main body 20.

### Advantages of This Embodiment

The controller 40 is configured or programmed to perform at least one of a control to move the robot main body based on the position of the tabletop 10 on the mobile cart 300, which is detected by the position detector 30, to attach the robot main body 20 to the tabletop 10 or a control to move the robot main body 20 based on the position of the mobile cart 300 without the tabletop 10 placed thereon, which is detected by the position detector 30, to place the tabletop 10 attached to the robot main body 20 on the mobile cart 300 to detach the tabletop 10 from the robot main body 20. Accordingly, the tabletop 10 can be attached and detached to and from the robot main body 20, and thus while treatment is performed on the patient P placed on the tabletop 10 attached to the robot main body 20, the next patient P to be treated can be positioned on the tabletop 10 on another mobile cart 300. Consequently, the throughput of the robot system 100 can be improved. Furthermore, the controller 40 can correct the amount of movement of the robot main body 20 even when the position of the tabletop 10 on the mobile cart 300 deviates from a position taught to the robot main body 20 in advance by performing a control to move the robot main body 20 based on the position of the tabletop 10 on the mobile cart 300, detected by the position detector 30. Consequently, the robot main body 20 can be appropriately attached to the tabletop 10. Furthermore, the controller 40 can correct the amount of movement of the robot main body 20 even when the position of the mobile cart 300 deviates from a position taught to the robot main body 20 in advance by performing a control to move the robot main body 20 based on the position of the mobile cart 300 detected by the position detector 30. Consequently, the tabletop 10 attached to the robot main body 20 can be appropriately on the mobile cart 300. Furthermore, the position detector 30 is on the robot main body 20, and thus the position detector 30 moves together with the robot main body 20. Therefore, unlike a case in which the position detector 30 is fixed at a position spaced apart from the robot main body 20, it is possible to reduce or prevent the possibility that the position detector 30 cannot detect an object to be detected, such as the tabletop 10 or the mobile cart 300, due to the object to be detected being hidden behind the robot main body 20. Consequently, at least one of a control to attach the robot main body 20 to the tabletop 10 or a control to detach the tabletop 10 from the robot main body 20 can be accurately performed.

The controller 40 is configured or programmed to perform both a control to attach the robot main body 20 to the tabletop 10 based on the position of the tabletop 10 detected by the position detector 30 and a control to detach the tabletop 10 from the robot main body 20 based on the position of the mobile cart 300 detected by the position detector 30. Accordingly, it is possible to appropriately attach the robot main body 20 to the tabletop 10, and appropriately place the tabletop 10 attached to the robot main body 20 on the mobile cart 300.

The position detector 30 includes the first imager 31 to image the tabletop 10 on the mobile cart 300, and the controller 40 is configured or programmed to perform a control to correct the position of the robot main body 20 with respect to the tabletop 10 based on the information captured by the first imager 31 to attach the robot main body 20. Accordingly, it is possible to detect a positional deviation of the tabletop 10 based on the information captured by the first imager 31. Consequently, the amount of movement of the robot main body 20 is corrected based on the detected positional deviation of the tabletop 10 such that the robot main body 20 can be appropriately attached.

The tabletop 10 includes the first markers 11, and the mobile cart 300 includes the notch 305. The tabletop 10 is placed on the Z1 side of the notch 305 of the mobile cart 300. The first markers 11 are on the lower surface 10a of the tabletop 10. The controller 40 is configured or programmed to perform a control to move the robot main body 20 to the Z2 side of the notch 305 of the mobile cart 300 on which the tabletop 10 is placed, cause the first imager 31 to image the first markers 11 on the lower surface 10a of the tabletop 10, and correct the position of the robot main body 20 with respect to the tabletop 10 based on the image of the first markers 11 captured by the first imager 31 to attach the robot main body 20 to the tabletop 10 on the mobile cart 300. Accordingly, when the robot main body 20 is attached to the tabletop 10 from the Z2 side of the tabletop 10, the first markers 11 on the lower surface 10a of the tabletop 10 can be easily imaged by the first imager 31 on the robot main body 20.

The position detector 30 includes the horizontal sensor 33 to detect the position of the object to be measured in the horizontal direction. The controller 40 is configured or programmed to perform a control to detect at least one of the tilt angle θ1 of the tabletop 10 with respect to the horizontal plane or the rotation angle θ2 of the tabletop 10 in the horizontal plane based on the detection result of the horizontal sensor 33, and correct the position of the robot main body 20 with respect to the tabletop 10 based on the image of the first markers 11 captured by the first imager 31 and at least one of the tilt angle θ1 or the rotation angle θ2 of the tabletop 10 to attach the robot main body 20 to the tabletop 10 on the mobile cart 300. The robot main body 20 moves toward the mobile cart 300 along a path that has been taught in advance in order to attach the tabletop 10. On the other hand, the mobile cart 300 may be positioned out of place relative to a predetermined position. Furthermore, a portion of the mobile cart 300 on which the tabletop 10 is may be tilted. In such a case, interference may occur between the robot main body 20, which moves to allow the tabletop 10 to be attached thereto, and the mobile cart 300 or the tabletop 10. Therefore, interference between the mobile cart 300 or the tabletop 10 and the robot main body 20 can be reduced or prevented by correcting the position of the robot main body 20 with respect to the tabletop 10 based on the tilt angle θ1 of the tabletop 10 with respect to the horizontal plane and the rotation angle θ2 of the tabletop 10 in the horizontal plane.

The mobile cart 300 includes the notch 305, and the pair of first detection target members 307a and 307b at both ends of the notch 305, respectively. The horizontal sensor 33 is operable to detect the positions of the first detection target members 307a and 307b in the horizontal direction. The controller 40 is configured or programmed to detect the height h1 of the lower end A1 and the height h2 of the lower end A2 of the pair of first detection target members 307a and 307b based on the detection results of the horizontal sensor 33, and detect the tilt angle θ1 of the tabletop 10 on the mobile cart 300 with respect to the horizontal plane based on the detected heights h1 and h2. A portion of the mobile cart 300, on which the tabletop 10 is, is tilted such that a difference occurs between the height h1 of the lower end A1 and the height h2 of the lower end A2. Therefore, the height h1 of the lower end A1 and the height h2 of the lower end A2 are detected based on the detection results of the horizontal sensor 33 such that the tilt angle θ1 of the tabletop 10 on the mobile cart 300 with respect to the horizontal plane can be easily detected.

The mobile cart 300 includes the pair of first detection target members 307a and 307b at both ends of the notch 305, respectively. The horizontal sensor 33 is operable to detect the positions of the first detection target members 307a and 307b in the horizontal direction. The controller 40 is configured or programmed to detect the interval L1 between the pair of first detection target members 307a and 307b based on the detection result of the horizontal sensor 33, and detect the rotation angle θ2 of the tabletop 10 in the horizontal plane based on the detected interval L1. The interval L1 between the pair of first detection target members 307a and 307b as viewed from the horizontal sensor 33 changes depending on the rotation angle θ2 of the mobile cart 300 in the horizontal plane. Therefore, the interval L1 between the pair of first detection target members 307a and 307b is detected based on the detection result of the horizontal sensor 33 such that the rotation angle θ2 of the tabletop 10 in the horizontal plane can be easily detected.

The position detector 30 includes the vertical sensor 34 to detect the distance to the tabletop 10 along the vertical direction. The controller 40 is configured or programmed to perform a control to attach the robot main body 20 to the tabletop 10 on the mobile cart 300 while detecting the distance to the tabletop 10 along the vertical direction with the vertical sensor 34. Accordingly, the robot main body 20 is moved while the distance to the tabletop 10 along the vertical direction is detected with the vertical sensor 34 such that interference between the robot main body 20 and the tabletop 10 can be easily reduced or prevented.

The mobile cart 300 includes the second detection target member 308. The position detector 30 further includes the horizontal sensor 33 to detect the position of the second detection target member 308 in the horizontal direction. The controller 40 is configured or programmed to perform a control to attach the robot main body 20 to the tabletop 10 on the mobile cart 300 while determining whether or not the relative positions of the robot main body 20 and the mobile cart 300 in the horizontal plane are normal based on the result of detection of the second detection target member 308 by the horizontal sensor 33 and while detecting the distance to the tabletop 10 along the vertical direction with the vertical sensor 34. Accordingly, it is possible to reduce or prevent the possibility that the operation to attach the robot main body 20 to the tabletop 10 on the mobile cart 300 is performed when the relative positions of the robot main body 20 and the mobile cart 300 in the horizontal plane are not normal.

The mobile cart 300 includes the second marker 306. The position detector 30 includes the second imager 32 to image the second marker 306 on the mobile cart 300 without the tabletop 10 placed thereon. The controller 40 is configured or programmed to perform a control to correct the position of the robot main body 20 with respect to the tabletop 10 based on the image of the second marker 306 captured by the second imager 32 to place the tabletop 10 attached to the robot main body 20 on the mobile cart 300 to detach the tabletop 10 from the robot main body 20. Accordingly, a positional deviation of the mobile cart 300 can be detected based on a positional deviation of the second marker 306 on the image captured by the second imager 32. Consequently, the amount of movement of the robot main body 20 is corrected based on the detected positional deviation of the mobile cart 300 such that the tabletop 10 attached to the robot main body 20 can be appropriately on the mobile cart 300.

The mobile cart 300 includes the notch 305. The second marker 306 is on the upper surface 300a of the mobile cart 300. The controller 40 is configured or programmed to perform a control to move the robot main body 20 to which the tabletop 10 has been attached to the Z1 side of the notch 305 of the mobile cart 300, cause the second imager 32 to image the second marker 306 on the mobile cart 300, and correct the position of the robot main body 20 with respect to the mobile cart 300 based on the image of the second marker 306 captured by the second imager 32 to place the tabletop 10 attached to the robot main body 20 on the mobile cart 300 to detach the tabletop 10 from the robot main body 20. Accordingly, when the tabletop 10 is placed on the mobile cart 300 from the Z1 side of the mobile cart 300, the second marker 306 on the upper surface 300a of the mobile cart 300 can be easily imaged by the second imager 32 on the robot main body 20.

The robot system 100 further includes the automatic tool changers 50 to attach and detach the tabletop 10 to and from the robot main body 20. The controller 40 is configured or programmed to control the automatic tool changers 50 to perform at least one of a control to attach the robot main body 20 to the tabletop 10 or a control to detach the tabletop 10 from the robot main body 20. Accordingly, the automatic tool changers 50 allow the tabletop 10 to be easily attached to and detached from the robot main body 20.

The robot main body 20 further includes the base member 22 attached to the distal end of the robot arm 21. The automatic tool changers 50 and the position detector 30 are on the base member 22. Accordingly, the degree of freedom in arranging the position detector 30 can be increased, as compared with a case in which the position detector 30 is directly attached to the robot arm 21. Furthermore, when the position detector 30 is directly attached to the robot arm 21, the robot arm 21 itself enters the detection range of the position detector 30 such that a blind spot may occur in the detection range of the position detector 30. Therefore, the position detector 30 is on the base member 22 attached to the distal end of the robot arm 21 such that it is possible to reduce or prevent the occurrence of a blind spot in the detection range of the position detector 30.

The robot system 100 further includes the torque sensor 60 on the robot main body 20 to detect a force applied to the robot main body 20 due to a load when the robot main body 20 is attached to the tabletop 10. The controller 40 is configured or programmed to perform a control to move the robot main body 20 based on the position of the tabletop 10 on the mobile cart 300 detected by the position detector 30, adjust the position of the robot main body 20 based on the force applied to the robot main body 20 detected by the torque sensor 60 while the robot main body 20 and the tabletop 10 contact each other, and attach the robot main body 20 to the tabletop 10 on the mobile cart 300. Consequently, even when the tabletop 10 cannot be appropriately attached and detached to and from the robot main body 20 by correcting the amount of movement of the robot main body 20 based on the detection result of the position detector 30, the tabletop 10 can be appropriately attached to and detached from the robot main body 20 by adjusting the position of the robot main body 20 based on the force applied to the robot main body 20 detected by the torque sensor 60.

### MODIFIED EXAMPLES

The embodiment disclosed this time must be considered as illustrative in all points and not restrictive. The scope of the present disclosure is not shown by the above description of the embodiment but by the scope of claims for patent, and all modifications (modified examples) within the meaning and scope equivalent to the scope of claims for patent are further included.

While the example in which the controller 40 corrects the position of the robot main body 20 based on the detection result of the position detector 30 in both a control to attach the robot main body 20 to the tabletop 10 and a control to detach the tabletop 10 from the robot main body 20 has been shown in the aforementioned embodiment, the present disclosure is not limited to this. For example, the controller 40 may correct the position of the robot main body 20 based on the detection result of the position detector 30 in only one of a control to attach the robot main body 20 to the tabletop 10 and a control to detach the tabletop 10 from the robot main body 20.

While the example in which the first imager 31 and the second imager 32 are two-dimensional cameras has been shown in the aforementioned embodiment, the present disclosure is not limited to this. For example, the first imager 31 and the second imager 32 may be three-dimensional cameras.

While the example in which the controller 40 detects the tile angle θ1 of the mounting table 301 of the mobile cart 300 and the rotation angle θ2 of the mounting table 301 around the axis along the Z direction based on the detection result of the horizontal sensor 33 has been shown in the aforementioned embodiment, the present disclosure is not limited to this. For example, the controller 40 may perform a control to correct the position of the robot main body 20 with respect to the tabletop 10 based only on the image of the first markers 11 captured by the first imager 31, without using the detection result of the horizontal sensor 33.

While the example in which the controller 40 performs a control to attach the robot main body 20 to the tabletop 10 placed on the mobile cart 300 while detecting the distance to the tabletop 10 along the vertical direction with the vertical sensor 34 has been shown in the aforementioned embodiment, the present disclosure is not limited to this. For example, the controller 40 may perform a control to attach the robot main body 20 to the tabletop 10 based only on the image of the first markers 11 captured by the first imager 31, without using the detection result of the vertical sensor 34.

While the example in which the controller 40 determines whether or not the relative positions of the robot main body 20 and the mobile cart 300 in the horizontal plane are normal based on the result of detection of the second detection target member 308 by the horizontal sensor 33 has been shown in the aforementioned embodiment, the present disclosure is not limited to this. For example, the controller 40 may perform a control to attach the robot main body 20 to the tabletop 10 based only on the image of the first markers 11 captured by the first imager 31, without using the result of detection of the second detection target member 308 by the horizontal sensor 33.

While the example in which the automatic tool changers 50 and the position detector 30 are arranged on the base member 22 has been shown in the aforementioned embodiment, the present disclosure is not limited to this. For example, the automatic tool changers 50 and the position detector 30 may be directly arranged on the robot arm 21.

While the example in which the present disclosure is applied to the robot system 100 including the tabletop 10, the robot main body 20, the controller 40, and the position detector 30 has been shown in the aforementioned embodiment, the present disclosure is not limited to this. For example, as shown in a modified example in FIG. 22, the present disclosure may be applied to a robot 400 including a robot main body 20, a controller 40, and a position detector 30.

While the example in which the plurality of first markers 11 are arranged has been shown in the aforementioned embodiment, the present disclosure is not limited to this. For example, three first markers 11 are arranged. Each first marker 11 has a circular shape, for example. The shapes of the plurality of first markers 11 may be the same as each other or may be different from each other. Furthermore, the colors of the plurality of first markers 11 may be the same as each other or may be different from each other. Moreover, each of the plurality of first markers 11 may have text or the like written thereon. The positions of the first markers 11 on the tabletop 10 are portions of the lower surface 10a of the tabletop 10 that are visible through the notch 305 (described below) of the mobile cart 300 when the tabletop 10 is placed on the mobile cart 300, for example.

While the example in which the controller 40 detects the tilt angle θ1 of the mounting table 301 based on the difference between the height h1 and the height h2 has been shown in the aforementioned embodiment, the present disclosure is not limited to this. For example, the height h1 of the lower end A1 of the first detection target member 307a and the height h2 of the lower end A2 of the first detection target member 307b may be different from each other in advance, and the controller 40 may detect the tilt angle θ1 of the mounting table 301 based on a change in the difference.

While the example in which the position detector 30 includes the vertical sensor 34 to detect the distance to the tabletop 10 along the vertical direction has been shown in the aforementioned embodiment, the present disclosure is not limited to this. For example, the position detector 30 may include a sensor to detect a distance along a direction intersecting with the vertical direction. The controller 40 may perform a control to attach the robot main body 20 to the tabletop 10 placed on the mobile cart 300 while detecting the distance to the tabletop 10 using the sensor that detects the distance along the direction intersecting with the vertical direction.

While the example in which the controller 40 corrects the position of the robot main body 20 with respect to the tabletop 10 based on the image of the first markers 11 captured by the first imager 31 has been shown in the aforementioned embodiment, the present disclosure is not limited to this. For example, the controller 40 may correct the position of the robot main body 20 with respect to the tabletop 10 based on information other than the image of the first markers 11 captured by the first imager 31.

While the example in which the inspection device 200 is an X-ray inspection device has been shown in the aforementioned embodiment, the present disclosure is not limited to this. For example, instead of the inspection device 200, the robot system 100 may be arranged in the vicinity of a treatment device.

While the example in which the mounting table 301 is supported by the lifters 302 has been shown in the aforementioned embodiment, the present disclosure is not limited to this. For example, the mounting table 301 may be supported by a support that does not include a lifting function, instead of the lifters 302.

While the example in which the notch 305 of the mounting table 301 has a rectangular shape has been shown in the aforementioned embodiment, the present disclosure is not limited to this. For example, the notch 305 may have a shape other than a rectangular shape.

While the example in which one second detection target member 308 is arranged has been shown in the aforementioned embodiment, the present disclosure is not limited to this. For example, a plurality of second detection target members 308 may be arranged.

The functionality of the elements disclosed herein may be implemented using circuitry or processing circuitry that includes general purpose processors, special purpose processors, integrated circuits, application specific integrated circuits (ASICs), conventional circuitry and/or combinations thereof that are configured or programmed to perform the disclosed functionality. Processors are considered processing circuitry or circuitry as they include transistors and other circuitry therein. In the present disclosure, the circuitry, units, or means are hardware that carries out the recited functionality or hardware that is programmed to perform the recited functionality. The hardware may be hardware disclosed herein or other known hardware that is programmed or configured to carry out the recited functionality. When the hardware is a processor that may be considered a type of circuitry, the circuitry, means, or units are a combination of hardware and software, and the software is used to configure the hardware and/or processor.

### ASPECTS

It will be appreciated by those skilled in the art that the exemplary embodiments described above are specific examples of the following aspects.

### Item 1

A robot system comprising:
a tabletop on a mobile cart to allow a patient to be placed thereon;
a robot main body including a robot arm to be detachably attached to the tabletop;
a position detector on the robot main body to detect at least one of a position of the tabletop on the mobile cart or a position of the mobile cart without the tabletop placed thereon; and
a controller; wherein
the controller is configured or programmed to perform at least one of:
   a control to move the robot main body based on the position of the tabletop on the mobile cart, which is detected by the position detector, to attach the robot main body to the tabletop; or
   a control to move the robot main body based on the position of the mobile cart without the tabletop placed thereon, which is detected by the position detector, to place the tabletop attached to the robot main body on the mobile cart to detach the tabletop from the robot main body.

### Item 2

The robot system according to item 1, wherein the controller is configured or programmed to perform both a control to attach the robot main body to the tabletop based on the position of the tabletop detected by the position detector and a control to detach the tabletop from the robot main body based on the position of the mobile cart detected by the position detector.

### Item 3

The robot system according to item 1 or 2, wherein
the position detector includes a first imager to image the tabletop on the mobile cart; and
the controller is configured or programmed to perform a control to correct a position of the robot main body with respect to the tabletop based on information captured by the first imager to attach the robot main body.

### Item 4

The robot system according to item 3, wherein
the tabletop includes a first marker;
the mobile cart includes a notch;
the tabletop is placed above the notch of the mobile cart;
the first marker is on a lower surface of the tabletop; and
the controller is configured or programmed to perform a control to:
   move the robot main body below the notch of the mobile cart on which the tabletop is placed;
   cause the first imager to image the first marker on the lower surface of the tabletop; and
   correct the position of the robot main body with respect to the tabletop based on an image of the first marker captured by the first imager to attach the robot main body to the tabletop on the mobile cart.

### Item 5

The robot system according to item 3 or 4, wherein
the position detector includes a horizontal sensor to detect a position of an object to be measured in a horizontal direction; and
the controller is configured or programmed to perform a control to:
   detect at least one of a tilt angle of the tabletop with respect to a horizontal plane or a rotation angle of the tabletop in the horizontal plane based on a detection result of the horizontal sensor; and
   correct the position of the robot main body with respect to the tabletop based on an image of the first marker captured by the first imager and at least one of the tilt angle or the rotation angle of the tabletop to attach the robot main body to the tabletop on the mobile cart.

### Item 6

The robot system according to item 5, wherein
the mobile cart includes:
   a notch; and
   a pair of first detection target members at both ends of the notch, respectively;
the horizontal sensor is operable to detect positions of the first detection target members in the horizontal direction; and
the controller is configured or programmed to:
   detect a height of a lower end of each of the pair of first detection target members based on the detection result of the horizontal sensor; and
   detect the tilt angle of the tabletop on the mobile cart with respect to the horizontal plane based on a detected height.

### Item 7

The robot system according to item 6, wherein
the mobile cart includes the pair of first detection target members at the both ends of the notch, respectively;
the horizontal sensor is operable to detect the positions of the first detection target members in the horizontal direction; and
the controller is configured or programmed to:
   detect an interval between the pair of first detection target members based on the detection result of the horizontal sensor; and
   detect the rotation angle of the tabletop in the horizontal plane based on a detected interval.

### Item 8

The robot system according to any one of items 1 to 7, wherein
the position detector includes a vertical sensor to detect a distance to the tabletop along a vertical direction; and
the controller is configured or programmed to perform a control to attach the robot main body to the tabletop on the mobile cart while detecting the distance to the tabletop along the vertical direction with the vertical sensor.

### Item 9

The robot system according to item 8, wherein
the mobile cart includes a second detection target member;
the position detector further includes a horizontal sensor to detect a position of the second detection target member in a horizontal direction; and
the controller is configured or programmed to perform a control to attach the robot main body to the tabletop on the mobile cart while determining whether or not relative positions of the robot main body and the mobile cart in a horizontal plane are normal based on a result of detection of the second detection target member by the horizontal sensor and while detecting the distance to the tabletop along the vertical direction with the vertical sensor.

### Item 10

The robot system according to any one of items 1 to 9, wherein
the mobile cart includes a second marker;
the position detector includes a second imager to image the second marker on the mobile cart without the tabletop placed thereon; and
the controller is configured or programmed to perform a control to correct a position of the robot main body with respect to the tabletop based on an image of the second marker captured by the second imager to place the tabletop attached to the robot main body on the mobile cart to detach the tabletop from the robot main body.

### Item 11

The robot system according to item 10, wherein
the mobile cart includes a notch;
the second marker is on an upper surface of the mobile cart; and
the controller is configured or programmed to perform a control to:
   move the robot main body to which the tabletop has been attached above the notch of the mobile cart;
   cause the second imager to image the second marker on the mobile cart; and
   correct a position of the robot main body with respect to the mobile cart based on the image of the second marker captured by the second imager to place the tabletop attached to the robot main body on the mobile cart to detach the tabletop from the robot main body.

### Item 12

The robot system according to any one of items 1 to 11, further comprising:
an automatic tool changer to attach and detach the tabletop to and from the robot main body; wherein
the controller is configured or programmed to control the automatic tool changer to perform at least one of a control to attach the robot main body to the tabletop or a control to detach the tabletop from the robot main body.

### Item 13

The robot system according to item 12, wherein
the robot main body further includes a base member attached to a distal end of the robot arm; and
the automatic tool changer and the position detector are on the base member.

### Item 14

The robot system according to any one of items 1 to 13, further comprising:
a torque sensor on the robot main body to detect a force applied to the robot main body due to a load when the robot main body is attached to the tabletop; wherein
the controller is configured or programmed to perform a control to:
   move the robot main body based on the position of the tabletop on the mobile cart detected by the position detector;
   adjust a position of the robot main body based on the force applied to the robot main body, which is detected by the torque sensor, while the robot main body and the tabletop contact each other; and
   attach the robot main body to the tabletop on the mobile cart.

### Item 15

A robot comprising:
a robot main body including a robot arm to be detachably attached to a tabletop on a mobile cart to allow a patient to be placed thereon;
a position detector on the robot main body to detect a position of the tabletop on the mobile cart or a position of the mobile cart without the tabletop placed thereon; and
a controller ; wherein
the controller is configured or programmed to perform at least one of:
   a control to move the robot main body based on the position of the tabletop on the mobile cart, which is detected by the position detector to attach the robot main body to the tabletop on the mobile cart; or
   a control to move the robot main body based on the position of the mobile cart without the tabletop placed thereon, which is detected by the position detector, to place the tabletop attached to the robot main body on the mobile cart to detach the tabletop from the robot main body.

1. A robot system comprising:
a tabletop on a mobile cart to allow a patient to be placed thereon;
a robot main body including a robot arm to be detachably attached to the tabletop;
a position detector on the robot main body to detect at least one of a position of the tabletop on the mobile cart or a position of the mobile cart without the tabletop placed thereon; and
a controller; wherein
the controller is configured or programmed to perform at least one of:
a control to move the robot main body based on the position of the tabletop on the mobile cart, which is detected by the position detector, to attach the robot main body to the tabletop; or
a control to move the robot main body based on the position of the mobile cart without the tabletop placed thereon, which is detected by the position detector, to place the tabletop attached to the robot main body on the mobile cart to detach the tabletop from the robot main body.

2. The robot system according to claim 1, wherein the controller is configured or programmed to perform both a control to attach the robot main body to the tabletop based on the position of the tabletop detected by the position detector and a control to detach the tabletop from the robot main body based on the position of the mobile cart detected by the position detector.

3. The robot system according to claim 1, wherein
the position detector includes a first imager to image the tabletop on the mobile cart; and
the controller is configured or programmed to perform a control to correct a position of the robot main body with respect to the tabletop based on information captured by the first imager to attach the robot main body.

4. The robot system according to claim 3, wherein
the tabletop includes a first marker;
the mobile cart includes a notch;
the tabletop is placed above the notch of the mobile cart;
the first marker is on a lower surface of the tabletop; and
the controller is configured or programmed to perform a control to:
move the robot main body below the notch of the mobile cart on which the tabletop is placed;
cause the first imager to image the first marker on the lower surface of the tabletop; and
correct the position of the robot main body with respect to the tabletop based on an image of the first marker captured by the first imager to attach the robot main body to the tabletop on the mobile cart.

5. The robot system according to claim 3, wherein
the position detector includes a horizontal sensor to detect a position of an object to be measured in a horizontal direction; and
the controller is configured or programmed to perform a control to:
detect at least one of a tilt angle of the tabletop with respect to a horizontal plane or a rotation angle of the tabletop in the horizontal plane based on a detection result of the horizontal sensor; and
correct the position of the robot main body with respect to the tabletop based on an image of the first marker captured by the first imager and at least one of the tilt angle or the rotation angle of the tabletop to attach the robot main body to the tabletop on the mobile cart.

6. The robot system according to claim 5, wherein
the mobile cart includes:
a notch; and
a pair of first detection target members at both ends of the notch, respectively;
the horizontal sensor is operable to detect positions of the first detection target members in the horizontal direction; and
the controller is configured or programmed to:
detect a height of a lower end of each of the pair of first detection target members based on the detection result of the horizontal sensor; and
detect the tilt angle of the tabletop on the mobile cart with respect to the horizontal plane based on a detected height.

7. The robot system according to claim 6, wherein
the mobile cart includes the pair of first detection target members at the both ends of the notch, respectively;
the horizontal sensor is operable to detect the positions of the first detection target members in the horizontal direction; and
the controller is configured or programmed to:
detect an interval between the pair of first detection target members based on the detection result of the horizontal sensor; and
detect the rotation angle of the tabletop in the horizontal plane based on a detected interval.

8. The robot system according to claim 1, wherein
the position detector includes a vertical sensor to detect a distance to the tabletop along a vertical direction; and
the controller is configured or programmed to perform a control to attach the robot main body to the tabletop on the mobile cart while detecting the distance to the tabletop along the vertical direction with the vertical sensor.

9. The robot system according to claim 8, wherein
the mobile cart includes a second detection target member;
the position detector further includes a horizontal sensor to detect a position of the second detection target member in a horizontal direction; and
the controller is configured or programmed to perform a control to attach the robot main body to the tabletop on the mobile cart while determining whether or not relative positions of the robot main body and the mobile cart in a horizontal plane are normal based on a result of detection of the second detection target member by the horizontal sensor and while detecting the distance to the tabletop along the vertical direction with the vertical sensor.

10. The robot system according to claim 1, wherein
the mobile cart includes a second marker;
the position detector includes a second imager to image the second marker on the mobile cart without the tabletop placed thereon; and
the controller is configured or programmed to perform a control to correct a position of the robot main body with respect to the tabletop based on an image of the second marker captured by the second imager to place the tabletop attached to the robot main body on the mobile cart to detach the tabletop from the robot main body.

11. The robot system according to claim 10, wherein
the mobile cart includes a notch;
the second marker is on an upper surface of the mobile cart; and
the controller is configured or programmed to perform a control to:
move the robot main body to which the tabletop has been attached above the notch of the mobile cart;
cause the second imager to image the second marker on the mobile cart; and
correct a position of the robot main body with respect to the mobile cart based on the image of the second marker captured by the second imager to place the tabletop attached to the robot main body on the mobile cart to detach the tabletop from the robot main body.

12. The robot system according to claim 1, further comprising:
an automatic tool changer to attach and detach the tabletop to and from the robot main body; wherein
the controller is configured or programmed to control the automatic tool changer to perform at least one of a control to attach the robot main body to the tabletop or a control to detach the tabletop from the robot main body.

13. The robot system according to claim 12, wherein
the robot main body further includes a base member attached to a distal end of the robot arm; and
the automatic tool changer and the position detector are on the base member.

14. The robot system according to claim 1, further comprising:
a torque sensor on the robot main body to detect a force applied to the robot main body due to a load when the robot main body is attached to the tabletop; wherein
the controller is configured or programmed to perform a control to:
move the robot main body based on the position of the tabletop on the mobile cart detected by the position detector;
adjust a position of the robot main body based on the force applied to the robot main body, which is detected by the torque sensor, while the robot main body and the tabletop contact each other; and
attach the robot main body to the tabletop on the mobile cart.

15. A robot comprising:
a robot main body including a robot arm to be detachably attached to a tabletop on a mobile cart to allow a patient to be placed thereon;
a position detector on the robot main body to detect a position of the tabletop on the mobile cart or a position of the mobile cart without the tabletop placed thereon; and
a controller; wherein
the controller is configured or programmed to perform at least one of:
a control to move the robot main body based on the position of the tabletop on the mobile cart, which is detected by the position detector to attach the robot main body to the tabletop on the mobile cart; or
a control to move the robot main body based on the position of the mobile cart without the tabletop placed thereon, which is detected by the position detector, to place the tabletop attached to the robot main body on the mobile cart to detach the tabletop from the robot main body.
